(19) United States
(12) Patent Application Publication
(10) Pub. No.: US 2004/0087966 A1
(43) Pub. Date: May 6, 2004

(54) Guidewire reel and related methods

(75) Inventor: Dennis M. McDevitt, Raleigh, NC (US)

Correspondence Address: RYAN KROMHOLZ & MANION, S.C., Post Office Box 26618, MILWAUKEE, WI, 53226, US

(73) Assignee: INCUMED INC.

(21) Appl. No.: 10/350,289

(22) Filed: Jan. 23, 2003

Related U.S. Application Data
(60) Provisional application No. 60/351,383, filed on Jan. 24, 2002.

(51) Int. Cl. A61F 11/00

(52) U.S. Cl. 606/108

(57) ABSTRACT

A modular guidewire reel allows the proximal end of a vascular guidewire to be captured, easily reeled up into a saline containing housing and keeps it readily available for subsequent use during a medical procedure.

### RELATED APPLICATIONS

This application claims the benefit of provisional Application Serial No. 60/351,383, filed Jan. 24, 2002.

### FIELD OF THE INVENTION

This invention relates to improved methods and devices for facilitating the storage and use of medical guidewires.

### BACKGROUND OF THE INVENTION

Many medical and diagnostic procedures require the insertion of an external instrument into a patient's body. Guidwires are employed in a variety of these procedures, e.g., in catheterization procedures, to aid in inserting and locating the instrument in a desired location.

One common technique employing the use of a guidewire is the Seldinger technique. In this technique, a hollow needle is inserted into a blood vessel. Next, a guidewire is inserted through the needle and into the vessel. The needle can then be withdrawn. An instrument, e.g., a catheter tube, is then advanced along the guidewire to the desired location and the desired procedure is performed.

Devices for storing and cleaning a guidewire are well known. Conventional guidewire storage devices are generally large, cumbersome bowls or basins with rims inside to keep the guidewire in a saline bath.

Conventional devices allow for numerous wires to get tangled, making it difficult to find and remove a wire in a timely manner. The awkward nature of using such devices often results in wires falling to the floor or otherwise becoming contaminated.

There remains a need to make the storage, cleansing, and access of guidewires during medical and surgical procedures more efficient.

### SUMMARY OF THE INVENTION

The invention a modular reel type device that allows the proximal (non-working) end of a guidewire to be captured and easily reeled into a housing. The housing may include a cleaning solution, e.g., physiologic saline, suitable for cleaning a guidewire. The device maintains the distal (working) end of the guidewire visible and readily available for subsequent use during a procedure.

According to one aspect of the invention, an apparatus for storing and dispensing an elongated member comprises a hub adapted to capture a proximal end of the elongated member and permits storage of the elongated member in a coiled configuration. A shell is sized and configured to receive the hub. An aperture sized and configured for passage of the elongated member transverses the circumferential margin of the shell.

According to another aspect of the invention, the hub has a peripheral groove and an annular groove. At least one cut-away opening is provided between the peripheral and annular grooves to permit passage of the proximal end of the elongated member from the peripheral groove to the annular groove for storage in the coiled configuration.

According to another aspect of the invention, rotation of the hub in a first direction advances the proximal end of the elongated member onto the hub to coil the hub for storage. The distal end of the elongated member remains conspicuously displayed to enable identification of the elongated member when coiled. Advancement of the distal end in the opposite direction uncoils the elongated member to dispense the elongated member for use. After the elongated member is dispensed, the proximal end can be advanced through the aperture to recapture the elongated member.

In one embodiment, the apparatus further comprises a post carried by the hub, a cover having a central opening to receive the post, and a wheel sized and configured to be received by the cover. The wheel includes a central opening sized and configured to mate with the post and a rotation mechanism to facilitate rotation of the wheel. Rotation of the wheel correspondingly rotates the hub. The rotation mechanism may be fingerhole and/or a knob.

Another aspect of the invention provides a method of engaging an elongated member. The method comprises passing a proximal end of the elongated member through an aperture that transverses the circumferential margin of a shell to capture the elongated member onto a hub, rotating the hub in a first direction to coil the elongated member onto the hub for storage, and pulling a distal end of the elongated member to rotate the hub in a second direction to dispense the elongated member for use.

In yet another aspect of the invention, the method further comprises after rotating the hub in the second direction, passing the proximal end of the elongated member through the aperture to recapture the elongated member onto the hub, and rotating the hub in the first direction to coil the elongated member onto the hub for storage.

### DESCRIPTION OF THE DRAWINGS

FIG. 1 is an exploded perspective view of a modular guidewire device embodying features of the invention.

FIG. 2 is a plan exploded view of the device shown in FIG. 1.

FIG. 3 is an assembled view of the device shown in FIG. 1.

FIG. 4 is a sectional view of the assembled device shown in FIG. 3.

FIG. 5 is an elevated partial sectional view of the assembled device shown in FIG. 3.

FIG. 6 is a perspective partial sectional view of the device shown in FIG. 3.

FIG. 7 is an elevational view of the device of FIG. 1, partially assembled, and illustrating a guidewire being thread through the device.

FIG. 8 is a perspective view of the device of FIG. 1, partially assembled, and illustrating a guidewire being thread through the device.

FIG. 9 is a top view of the device of FIG. 1, partially assembled, and illustrating a guidewire being thread through the hub.

FIG. 10 is a perspective view of the hub component of the device of FIG. 1, and illustrating a guidewire coiled around the hub.

### DETAILED DESCRIPTION

Although the disclosure hereof is detailed and exact to enable those skilled in the art to practice the invention, the physical embodiments herein disclosed merely exemplify the invention that may be embodied in other specific structure. While the preferred embodiment has been described, the details may be changed without departing from the invention, which is defined by the claims.

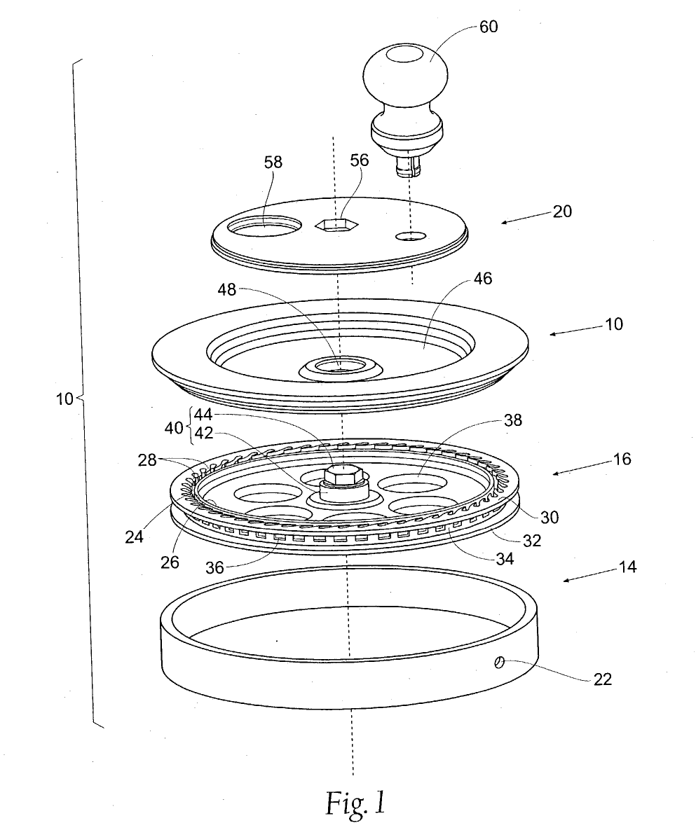

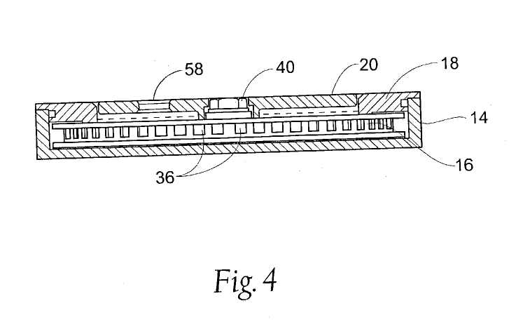

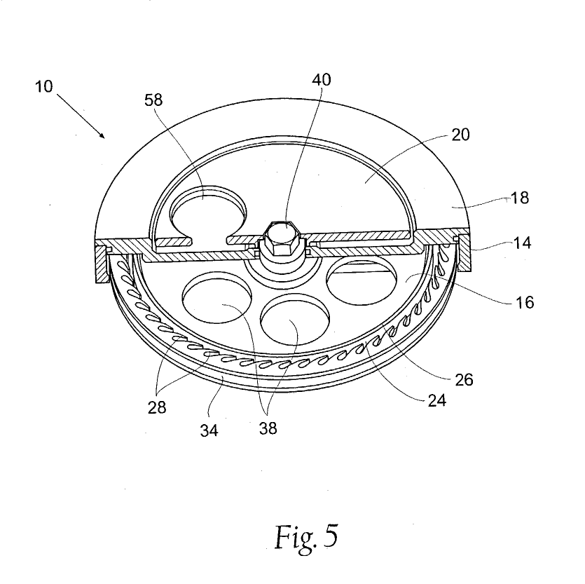

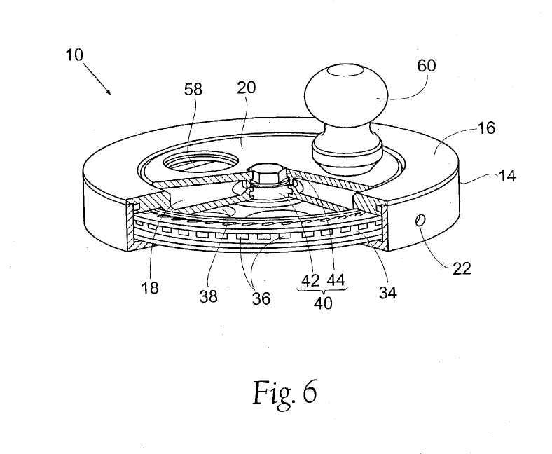

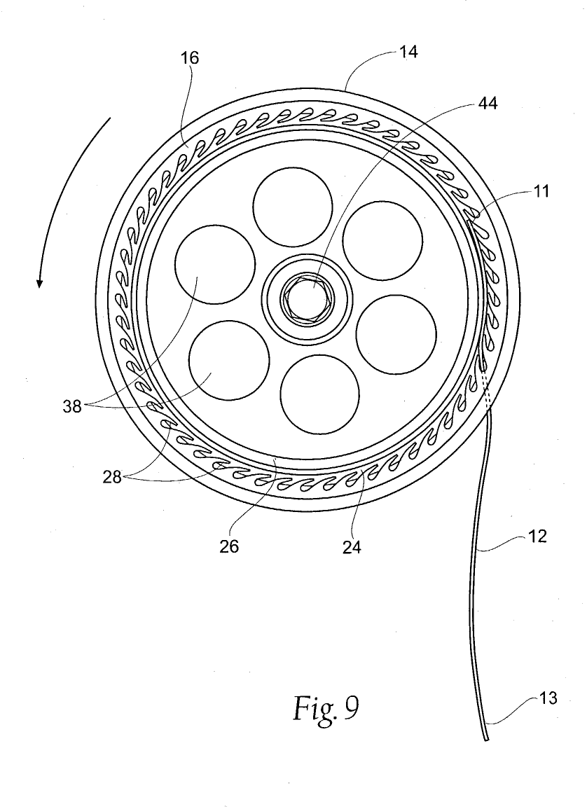

The FIGS. 1-10 show the cooperating components of a modular guidewire reel device 10 embodying features of the invention. The device 10 allows the proximal 11, i.e., non-working, end of a guidewire 12 to be captured and easily reeled up for storage (see FIG. 9). Desirably, the distal end 13, i.e., working end, of the guidewire 12 remains exposed, permitting easy access. The device 10 thus keeps the reeled proximal end 11 readily available for subsequent use during the procedure. The device 10 comprises a shell 14, a hub 16, a cover 18, and a wheel 20.

As best seen in FIG. 1, the shell 14 is a solid, bowl-like member adapted to hold a solution suitable for cleaning a guidewire 12, e.g., physiologic saline. The shell 14 includes an aperture 22 that transverses the circumferential margin and accommodates passage of a guidewire 12. A funnel-like seal on the inside of the shell 14 can cover the aperture 22 to prevent leakage of cleaning solution from the shell 14 (not shown).

The hub 16 has a reel-like configuration and is adapted to rest within the shell 14. The hub 16 includes an annular groove 24 that defines an interior wall 26.

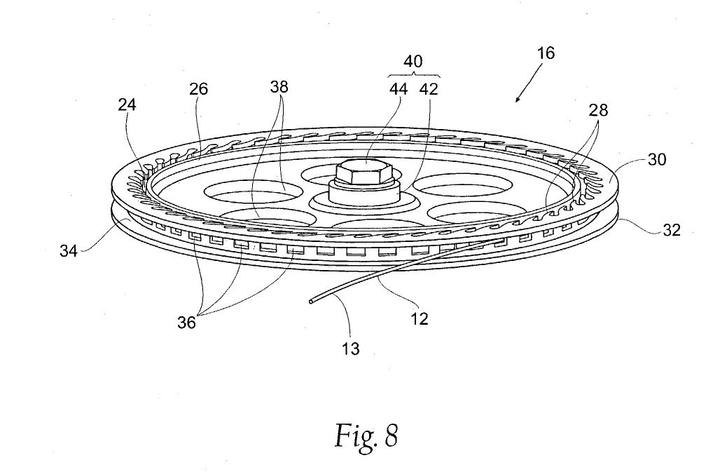

An upper rim 30 and a lower rim 32 surround the circumferential margin of the hub 16 to define a peripheral groove 34. With reference to FIGS. 8-10, a series of U-shaped cut-away openings 36 between the annular groove 24 and the peripheral groove 34 define a series of teeth 28 and serve to permit passage of the proximal end 11 of the guidewire 12 into the annular groove 24 as it is threaded through the aperture 22. The angle of the cut-away 36, the curve of the annular groove 24, and the stiffness of the guidewire 12 together create pinch points on the guidewire 12 that engage and capture the proximal end 11 of the guidewire 12 within the annular groove 24. In an alternative embodiment, the cut-away 36 is a generally round hole that passes into the annular groove 24 at an angle (not shown).

The size and configuration of the hub 16 and openings 36, along with the number and frequency of openings 36, can be varied to accommodate the capture of guidewires 12 of varying diameter, length, and stiffness.

A series of holes 38 is desirably provided in the bottom of the hub 16 to permit the cleaning solution to flow across the surface of the hub 16. In a representative embodiment, as seen in FIG. 1, the hub 16 includes a series of six holes 38 radially spaced from the center of the hub 16. It is to be understood that the number and placement of the holes 38 can vary.

With reference again to FIG. 1, the hub 16 includes an upstanding central post 40 to secure placement of the cover 18 and the wheel 20. The post 40 includes an annular collar 42 and a rotation link 44.

The cover 18 is a plate-like structure having a recessed central portion defining a chamber 46 that receives the wheel 20. The chamber 46 includes a central opening 48 that engages the annular collar 42.

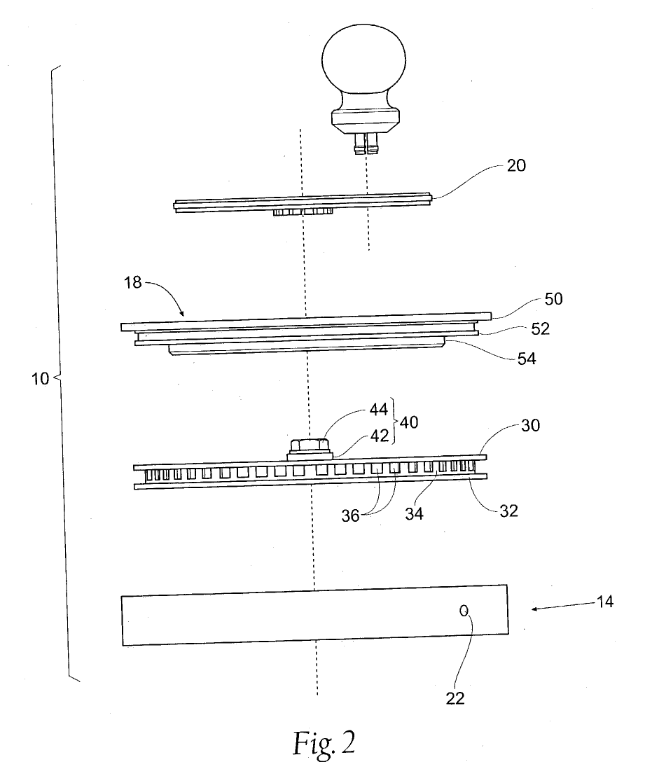

In a representative embodiment, as best seen in FIG. 2, the cover 18 includes a tapered three-tier configuration. In this arrangement, the first tier 50 is fixed to the shell 14. The second tier 52 rests on the upper rim 30 of the hub 16. The third tier 54 rests against the interior wall 26 of the hub 16. This arrangement serves to secure the cover 18 and the hub 16 within the shell 14.

The wheel 20 includes a central opening 56 -7 that is configured to mate with the rotation link 44. In a representative embodiment, as seen in FIG. 1, the central opening 56 has a hexagonal configuration that mates with a corresponding hexagonal configuration of the rotation link 44.

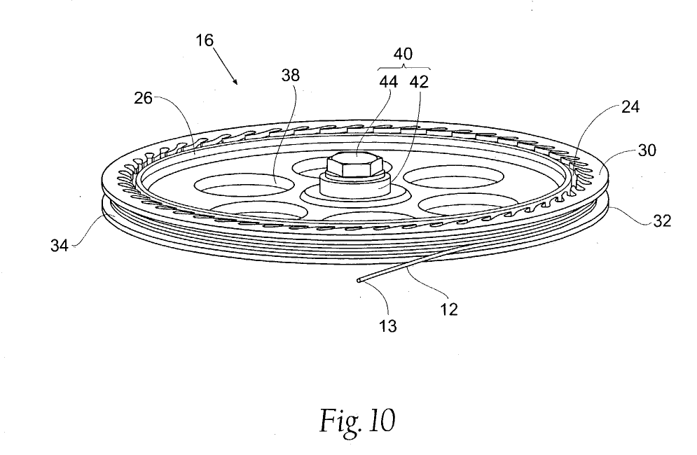

Rotation of the wheel 20 in a given direction, indicated by arrow in FIG. 9, simultaneously correspondingly rotates the hub 16. After the proximal end 11 of the guidewire 12 is engaged by the hub 16, rotation of the hub 16 advances the proximal 11 end of the guidewire 12 onto the hub 16 and coils the guidewire 12 about the peripheral groove 34. The rotation can be continued until the guidewire 12 is coiled within the peripheral groove 34 of the hub 16 (see FIG. 10). Desirably, a portion of the distal 13 end of the guidewire 12, e.g., several centimeters, remains exposed. Thus, the distal end 13 can remain conspicuously displayed to ease identification of guidewires 12 when more than one guidewire 12 is used.

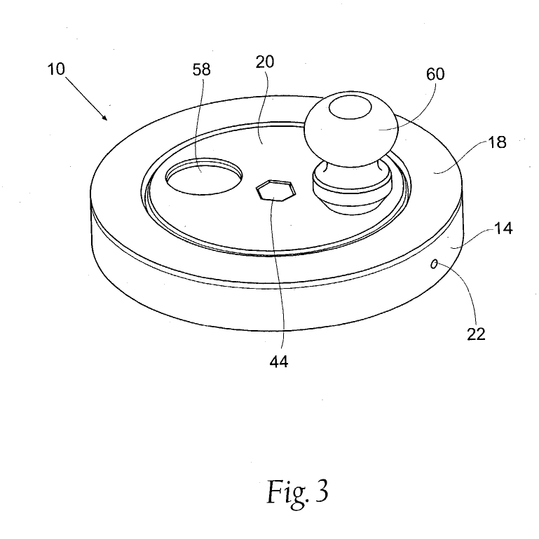

The wheel 20 may include either or both of a fingerhole 58 and a knob 60 (see, e.g., FIG. 3). The fingerhole 58 and knob 60 permit easy grasping and rotation of the wheel 20. If a preferred embodiment, the knob is selectively removable, as seen in FIG. 1, to facilitate storage and stacking.

Pulling or otherwise advancing the distal end 13 of the guidewire 12 rotates the hub 16 in the opposite direction, thereby unreeling and releasing the guidewire 12.

The device 10 is desirably made of any suitable plastic by conventional molding techniques, making it suitable for use as a sterile, single-use, disposable unit (see, e.g., FIG. 3). It is apparent, however, that suitable metals or other materials could also be employed.

The device 10 is sized for placement on a procedure table or mayo stand and is suitable for stacking.

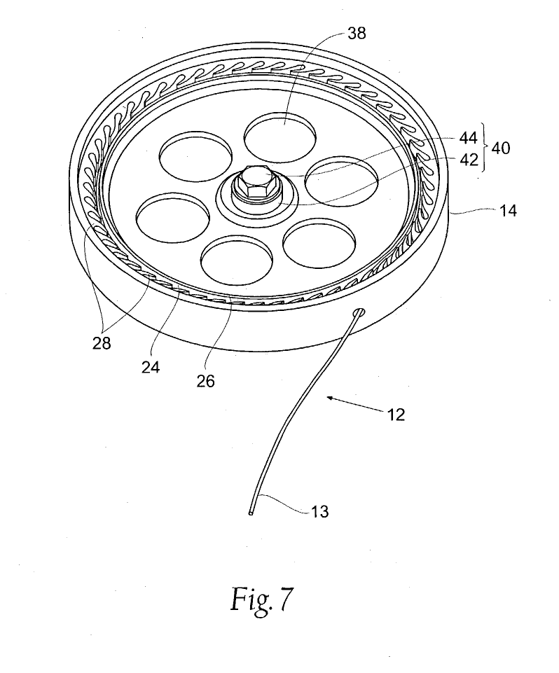

The device 10 may be supplied along with a separate guidewire 12 of specified length as a kit in an assembled, sterile condition. In use, the practitioner fills the shell 14 with physiologic saline or other suitable cleaning solution, e.g., by injection with a syringe through the aperture 22 (not shown). As FIGS. 7-10 detail, the practitioner then inserts the proximal 11 end of the guidewire 12 through the aperture 22 and a U-shaped cut-away 36, thereby catching the guidewire 12 on the hub 16. The hub 16 is then rotated by rotating the wheel 20 in a given direction to reel the guidewire 12 onto the hub 16 within the peripheral groove 34. A portion of the distal 13 end of the guidewire 12 is left exposed. To release the reeled guidewire 12, the practitioner pulls on the distal 13 end of the guidewire 12. Pulling the distal 13 end of the guidewire 12 rotates the hub 16 in the opposite direction, thereby unreeling and releasing the guidewire 12.

Alternatively, the device 10 may be supplied pre-filled with cleaning solution along with a guidewire 12 as a kit in an assembled, sterile, and ready-to-use condition.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. An apparatus for storing and dispensing an elongated member, the apparatus comprising
a hub adapted to capture a proximal end of the elongated member and permitting storage of the elongated member in a coiled configuration,
a shell sized and configured to receive the hub, and,
an aperture transversing a circumferential margin of the shell, the aperture being sized and configured for passage of the elongated member.

2. An apparatus for storing and dispensing an elongated member, the apparatus comprising
a hub having a peripheral groove and an annular groove,
at least one cut-away opening between the peripheral and annular grooves permitting passage of a proximal end of an elongated member from the peripheral groove to the annular groove for storage in a coiled configuration,
a shell sized and configured to receive the hub, and
an aperture transversing a circumferential margin of the shell, the aperture being sized and configured for passage of the elongated member.

3. An apparatus as in claim 1 or 2
wherein the elongated member is a guidewire.

4. An apparatus as in claim 1 or 2
wherein the shell is adapted to hold a cleaning solution.

5. An apparatus as in claim 4
wherein the cleaning solution is saline.

6. An apparatus as in claim 1 or 2
wherein rotation of the hub in a first direction advances the proximal end of the elongated member onto the hub to coil the elongated member onto the hub for storage.

7. An apparatus as in claim 6
wherein the distal end of the elongated member remains conspicuously displayed to enable identification of the elongated member when coiled.

8. An apparatus as in claim 7
wherein advancement of the distal end of the elongated member in the opposite direction uncoils the elongated member to dispense the elongated member for use.

9. An apparatus as in claim 8
wherein, after the elongated member is dispensed, the proximal end can be advanced through the aperture to recapture the elongated member.

10. An apparatus as in claim 1, further comprising
a post carried by the hub,
a cover having a central opening to receive the post, and
a wheel sized and configured to be received by the cover and including a central opening sized and configured to mate with the post and a rotation mechanism to facilitate rotation of the wheel, whereby rotation of the wheel correspondingly rotates the hub.

11. An apparatus as in claim 10
wherein the rotation mechanism is a fingerhole.

12. An apparatus as in claim 10
wherein the rotation mechanism is a knob.

13. A method of engaging an elongated member comprising
passing a proximal end of the elongated member through an aperture that transverses the circumferential margin of a shell to capture the elongated member onto a hub,
rotating the hub in a first direction to coil the elongated member onto the hub for storage, and
pulling a distal end of the elongated member to thereby rotate the hub in a second direction to dispense the elongated member for use.

14. A method as in claim 13, further comprising
after pulling the distal end of the elongated member to dispense the elongated member for use, passing the proximal end of the elongated member through the aperture to recapture the elongated member onto the hub, and
rotating the hub in the first direction to coil the elongated member onto the hub for storage.
